(19) United States
(12) Patent Application Publication
(10) Pub. No.: US 2024/0075301 A1
(43) Pub. Date: Mar. 7, 2024

(54) An Implantable Medical Device Configured to Provide an Intra-Cardiac Function

(71) Applicant: BIOTRONIK SE & Co. KG, Berlin (DE)

(72) Inventors: Christopher JONES, Oregon City, OR (US); Daniel YOUNG, Portland, OR (US); Madeline Anne MIDGETT, Portland, OR (US); R. Hollis WHITTINGTON, Portland, OR (US)

(73) Assignee: BIOTRONIK SE & Co., KG, Berlin (DE)

(21) Appl. No.: 18/262,585

(22) PCT Filed: Feb. 3, 2022
(86) PCT No.: PCT/EP2022/052525

§ 371 Date: Jul. 24, 2023

Related U.S. Application Data
(60) Provisional application No. 63/148,196, filed on Feb. 11, 2021.

(30) Foreign Application Priority Data
Mar. 25, 2021 (EP) 21164827.4

(51) Int. Cl. A61N 1/37; A61N 1/365; A61N 1/375

(52) U.S. Cl. CPC A61N 1/3704; A61N 1/365; A61N 1/3756

(57) ABSTRACT

An implantable medical device configured to provide for an intracardiac function comprises a body, a sensor arrangement arranged on the body and configured to receive a cardiac sense signal, and a processing circuitry operatively connected to the sensor arrangement. The processing circuitry is configured to process the cardiac sense signal received using the sensor arrangement to detect, in a cardiac cycle, a first atrial event candidate based on a comparison of the cardiac sense signal with a sense threshold, to determine a modified sense threshold, to monitor, by comparing the cardiac sense signal to said modified sense threshold following the detection of the first atrial event candidate, whether in the same cardiac cycle a second atrial event candidate is detected.

### CROSS-REFERENCE TO RELATED APPLICATIONS

This application is the United States National Phase under 35 U.S.C. § 371 of PCT International Patent Application No. PCT/EP2022/052525, filed on Feb. 3, 2022, which claims the benefit of European Patent Application No. 21164827.4, filed on Mar. 25, 2021 and U.S. Provisional Patent Application No. 63/148,196, filed on Feb. 11, 2021, the disclosures of which are hereby incorporated by reference herein in their entireties.

### TECHNICAL FIELD

The present invention generally relates to an implantable medical device for providing an intracardiac function, in particular a pacing function such as ventricular pacing, specifically a VDD pacing.

### BACKGROUND

In an implantable medical device, e.g., in the shape of a leadless pacemaker device or a cardiac stimulation device using a subcutaneously implanted pulse generator and one or multiple leads extending into a patient's heart, it may be desirous to provide stimulation in a ventricle of the patient's heart, e.g., in the right ventricle, in synchrony with an atrial activity.

For this, ventricular pacing shall take into account atrial sense signals to control the ventricular pacing based on atrial events indicative of atrial activity, for example, in a so-called VDD pacing mode.

In recent years, leadless pacemakers have received increasing attention. Leadless pacemakers, in contrast to pacemakers implanted subcutaneously using leads extending transvenously into the heart, avoid leads in that the pacemaker device itself is implanted into the heart, the pacemaker having the shape of a capsule for implantation into cardiac tissue, in particular the right ventricle. Such leadless pacemakers exhibit the inherent advantage of not using leads, which can reduce risks for the patient involved with leads transvenously accessing the heart, such as the risk of pneumothorax, lead dislodgement, cardiac perforation, venous thrombosis and the like.

A leadless pacemaker or a lead of a stimulation device may specifically be designed for implantation in the right ventricle and, in this case, during implant is placed, e.g., in the vicinity of the apex of the right ventricle. Ventricular pacing may, for example, be indicated in case a dysfunction at the AV node occurs, but the sinus node function is intact and appropriate. In such a case in particular a so-called VDD pacing may be desired, involving ventricular pacing with atrial tracking and hence requiring sensing of atrial activity in order to a pace at the ventricle based on intrinsic atrial contractions.

A VDD pacing is in particular motivated by patient hemodynamic benefits of atrioventricular (AV) synchrony by utilizing an appropriate sinus node function to trigger ventricular pacing, potentially allowing to maximize ventricular preload, to limit AV valve regurgitation, to maintain low mean atrial pressure, and to regulate autonomic and neurohumoral reflexes.

Publications have explored solutions to use modalities to detect mechanical events of atrial contractions, including the sensing of motion, sound and pressure (see, for example, U.S. Publication No. 2018/0021581 A1 disclosing a leadless cardiac pacemaker including a pressure sensor and/or an accelerometer to determine an atrial contraction timing). As mechanical events generally exhibit a small signal volume, signal detection based on mechanical events, for example, motion, sound or pressure, may be difficult to sense, in particular when the implantable medical device is placed in the ventricle and hence rather far removed from the atrium of which contractions shall be sensed. In addition, wall motion and movement of blood generated by atrial contractions may not be directly translated to the ventricle, and cardiac hemodynamic signals, such as motion, heart sounds and pressure, are likely affected by external factors such as posture and patient activity.

The present disclosure is directed toward overcoming one or more of the above-mentioned problems, though not necessarily limited to embodiments that do.

### SUMMARY

It is an objective to provide an implantable medical device and a method for operating an implantable medical device allowing, in particular, for ventricular pacing with atrioventricular synchrony, hence requiring a reliable sensing of atrial events in order to provide for a ventricular pacing based on such atrial events.

Such desires are addressed by an implantable medical device configured to provide for an intracardiac function having the features of claim 1.

In one aspect, an implantable medical device configured to provide for an intracardiac function comprises a body, a sensor arrangement on the body and configured to receive a cardiac sense signal, and processing circuitry operatively connected to the sensor arrangement. The processing circuitry herein is configured to process the cardiac sense signal received using the sensor arrangement to detect, in a cardiac cycle, a first atrial event candidate based on a comparison of the cardiac sense signal with a sense threshold, to determine a modified sense threshold, to monitor, by comparing the cardiac sense signal to said modified sense threshold following the detection of the first atrial event candidate, whether in the same cardiac cycle a second atrial event candidate is detected.

If an implantable medical device is implanted in a ventricle, for example, the right ventricle, of the patient's heart to provide for a function (such as a sensing function or a pacing function) in the ventricle, signals relating to atrial activity received by the sensor arrangement of the implantable medical device are in the far-field and hence may have a small signal amplitude and may be noisy. This may lead to a false detection of noise prior to an actual atrial event. For example, a false detection may occur before the true P wave in an intracardiac electrogram. If such a false detection is erroneously identified as a valid atrial event for a cardiac cycle, an atrial-ventricular delay may be falsely started, and a pacing action may be triggered based on incorrect timing. This may cause a loss of atrial-ventricular synchrony, in particular if false detection of atrial events occurs over multiple cardiac cycles.

For this reason, a scheme is proposed to verify an atrial event candidate detected in a cardiac cycle by monitoring a cardiac sense signal for another atrial event candidate in the same cardiac cycle. If it is found that another atrial event candidate subsequent to the first atrial event candidate likely represents the true atrial event, for example, the subsequent detection represents a true P wave in an intra-cardiac electrogram, the first atrial event candidate may be discarded, and the other atrial event candidate (detected after the first atrial event candidate in the same cardiac cycle) may be assumed as the true, valid atrial event.

For verifying and potentially correcting the occurrence of an atrial event, first an atrial event candidate is detected based on a comparison of a received cardiac sense signal with a sense threshold. Subsequent to the occurrence of the first atrial event candidate, a modified sense threshold is determined, for example, based on an amplitude value as determined for the first atrial event candidate, the modified sense threshold in one embodiment being larger than the initial sense threshold based on which the first atrial event candidate has been determined. Using the modified sense threshold, in a time period following the first atrial event candidate, the cardiac signal is monitored to determine whether one or multiple subsequent atrial event candidates occur. Based on the different atrial event candidates, it then may be identified which of the atrial event candidates likely represents the true atrial event, in particular an event relating to a P wave in an intra-cardiac electrogram signal. The true atrial event may be identified as valid atrial event for the cardiac cycle, used to generate an atrial sense marker within the device, and may be used to trigger a pacing action.

By monitoring for an additional atrial event candidate following a first atrial event candidate in the same cardiac cycle, it becomes possible to override the first atrial event candidate and correct any values or time intervals derived from the first atrial event candidate. False detections of events prior to the actual atrial event, namely prior to the actual P wave in an intra-cardiac electrogram signal, hence may be avoided, which may improve reliability of atrial detection in order to maintain atrial-ventricular synchrony.

The sensor arrangement in particular may be formed by an electrode arrangement of one or multiple electrodes arranged on the body. Hence, by means of the sensor arrangement electrical signals may be received, such electrical signals representing intracardiac electrogram recordings and hence being indicative of cardiac activity.

In another embodiment, the sensor arrangement may be configured for sensing cardiac sense signals in the shape of pressure signals, acoustic signals, ultrasound signals, motion signals, and/or impedance signals.

In one embodiment, the body of the implantable medical device may be formed by a lead which is connectable to a generator of the implantable medical device. In this case, the generator may be implanted into a patient, for example, subcutaneously remote from the heart, the lead forming the body extending from the generator into the heart such that the body with the sensor arrangement arranged thereon is placed in the heart, for example, within the right ventricle in order to engage with tissue at the right ventricle.

In another embodiment, the body may be formed by a housing of a leadless pacemaker device. In this case, the implantable medical device is formed as a leadless device, which does not comprise leads extending from a location outside of the heart into the heart for providing for a stimulation and/or sensing within the heart. The housing of the leadless pacemaker device may be placed on tissue with a distal end formed by the housing, the sensor arrangement, e.g., being placed (at least in part) on or in the vicinity of the distal end and engaging with tissue when placing the leadless pacemaker device on tissue with its distal end.

If the implantable medical device is a leadless pacemaker device, the housing provides for an encapsulation of the implantable medical device, the implantable medical device including all required components for autonomous operation, such as the processing circuitry, an energy storage such as a battery, electric and electronic circuitry and the like, within the housing. The housing is fluid-tight such that the implantable medical device may be implanted into cardiac tissue and may be kept in cardiac tissue over an extended period of time to provide for a long-term, continuous cardiac pacing operation.

In one embodiment, the processing circuitry is configured to identify the first atrial event candidate as a valid atrial event for the cardiac cycle if no second atrial event candidate is detected. Alternatively, if a second atrial event candidate is detected within the monitoring interval subsequent to the first atrial event candidate, the processing circuitry may be configured to identify the second atrial event candidate as a valid atrial event for the cardiac cycle. Hence, based on whether a second atrial event candidate after the first atrial event candidate is detected, the first atrial event candidate is overridden (in case a second atrial event candidate is detected) or is maintained and assumed as a true atrial event (in case no second atrial event candidate is detected following the first atrial event candidate).

The steps may be repeated. Hence, following a detection of a second atrial event candidate, an additional monitoring may take place in which the cardiac signal is monitored for further atrial event candidates, such that also the second atrial event candidate may be overridden. In particular, the processing circuitry may be configured to determine a new modified sense threshold if a second atrial event candidate is detected, and to monitor, by comparing the cardiac sense signal to said new modified sense threshold following the detection of the second atrial event candidate, whether in the same cardiac cycle a third atrial event candidate is detected. If a third atrial event candidate is detected, this may be assumed as the true atrial event and may be identified as valid.

In one embodiment, the processing circuitry may be configured to detect the first atrial event candidate based on a crossing of the sense threshold by the cardiac sense signal. Generally, the cardiac sense signal, for example, an intra-cardiac electrogram signal as picked up by an electrode arrangement of the implantable medical device, is compared to the sense threshold, which may be determined based on peak amplitude values as determined in one or multiple previous cardiac cycles. Based on the comparison, in particular based on the detection of a threshold crossing, the first atrial event candidate is identified, and following the first atrial event candidate it is monitored whether one or multiple further atrial event candidates occur.

In one embodiment, the processing circuitry is configured to determine the modified sense threshold based on a peak amplitude determined for the first atrial event candidate. Namely, once the first atrial event candidate is detected based on a comparison of the sense signal to the initial sense threshold, in particular based on a threshold crossing of the initial sense threshold by the cardiac sense signal, a scheme for determining a peak amplitude associated with the first atrial event candidate is carried out. Based on the peak amplitude determined in this manner, then, the modified sense threshold for the detection of a second atrial event candidate is determined, the modified sense threshold in particular being larger than the initial sense threshold.

In another embodiment, the modified sense threshold may be set based on the first sense threshold rather than the first atrial detection peak. For example, the modified sense threshold may be set at some constant above the first sense threshold, for example, by adding a constant value to the sense threshold or by multiplying the sense threshold by a constant factor. In this case a tracking of the peaks of the candidate atrial events may not be necessary.

In one embodiment, the processing circuitry is configured to determine the peak amplitude in a peak detection window following the first atrial event candidate. The first atrial event candidate may, for example, be assumed as that point in time at which a threshold crossing of the initial sense threshold is (reliably) identified. In one embodiment, the first atrial event candidate may be detected if one signal value of the cardiac sense signal is larger than the sense threshold. In another embodiment, the first atrial event candidate may be assumed as that point in time at which a predefined number of samples of the cardiac sense signal are detected which are larger than the sense threshold. At the time of the first atrial event candidate a peak detection window is started, and within the peak detection window the cardiac sense signal is tracked to determine the maximum of the cardiac sense signal within the peak detection window. This maximum value may be assumed as the peak amplitude associated with the first atrial event candidate. Making use of the peak amplitude, then, the modified sense threshold may be set, for example, by multiplying the peak amplitude by a factor such that the modified sense threshold, for example, is larger than the peak amplitude associated with the first atrial event candidate.

In one embodiment, the processing circuitry is configured to set, if a second atrial event candidate is detected, the peak amplitude anew according to a peak amplitude value associated with the second atrial event candidate.

At the occurrence of a second atrial event candidate, for example, another peak detection window may be started in order to determine the peak amplitude value anew within the peak detection window associated with the second atrial event candidate. Hence, again, within the peak detection window following the second atrial event candidate the cardiac sense signal is tracked and the maximum value of the cardiac sense signal within the peak detection window is assumed as the peak amplitude.

In another embodiment, the peak amplitude may be set to the signal value of the cardiac sense signal at the time of the second atrial event candidate, i.e., at the time of the threshold crossing of the modified sense threshold.

Whereas the determination of the peak amplitude by means of a second peak detection window following the second atrial event candidate may have the advantage of an exact determination of the peak amplitude value, the determination of the peak amplitude by setting the peak amplitude to the signal value at the time of the second atrial event candidate may have the advantage of fast processing in that the peak amplitude is immediately obtained at the time of detecting a second atrial event candidate.

Based on the peak amplitude of the identified valid atrial event, a sense threshold for another subsequent cardiac cycle may be determined (wherein it also is possible to set the sense threshold based on the previous sense threshold, in which case no tracking of the peak amplitude may even be required).

In one embodiment, the processing circuitry is configured to start, at the time of detection of the first atrial event candidate, a detection hold-off period and to delay monitoring the cardiac sense signal for the detection of a second atrial event candidate until after lapse of said detection hold-off period. Hence, subsequent to the first atrial event candidate, for a predefined period of time (i.e., the detection hold-off period) no detection of a further, second atrial event candidate is possible. The monitoring for the second atrial event candidate starts only after lapse of the detection hold-off period.

The detection hold-off period generally should be long enough such that multiple detections due to a single atrial event are prevented.

At the time of detection of a second atrial event candidate, a (second) detection hold-off period may be started, such that following the second atrial event candidate monitoring for further atrial event candidates takes place only after lapse of the (second) detection hold-off period.

In one embodiment, the processing circuitry is configured to start, at the time of detection of the first atrial event candidate, an atrial-ventricular delay period and to stop monitoring for the detection of a second atrial event candidate after lapse of the atrial-ventricular delay period. The atrial-ventricular delay period indicates a time period after which, following an atrial event, a ventricular pace is expected to be triggered. If within the atrial-ventricular delay period no further atrial event candidate occurs, it can be assumed that the (first) atrial event candidate represents a true, valid atrial event, such that monitoring for further atrial event candidates can be stopped.

In case no ventricular activity is detected after the atrial-ventricular delay period, a pacing action may be triggered in order to stimulate ventricular activity.

In another embodiment, the processing circuitry may be configured to start, at the time of detection of said second atrial event candidate, the atrial-ventricular delay period anew. If within the atrial-ventricular delay period as started at the time of detection of the first atrial event candidate a subsequent second atrial event candidate is detected, it is assumed that the first atrial event candidate does not represent the true, valid atrial event. Hence, the atrial-ventricular delay period is started anew at the time of detection of the second atrial event candidate, wherein monitoring for further atrial event candidates may also be started.

Using the peak amplitude as determined for the valid atrial event (which may be, e.g., the first atrial event candidate or a subsequently detected further atrial event), the processing circuitry may be configured to compute the sense threshold for detecting a (first) atrial event candidate in a subsequent cardiac cycle. In particular, the processing circuitry may be configured to update the sense threshold using an average threshold reference and a percentage ratio according to the formula

ST=PC·ATR(t),

where ST is the current sense threshold, PC is a percentage ratio, and ATR(t) is the current average threshold reference for cycle t. The percentage ratio may lie, for example, in the range between 0% and 100% and may be programmable.

The average threshold reference may, for example, be computed as a mean value of peak amplitudes for a predefined number of cardiac cycles in which atrial events have been detected, for example, a number in between two to six cardiac cycles.

In one embodiment, the processing circuitry comprises a first processing channel having a first gain for processing a first processing signal derived from signals received via the sensor arrangement and a second processing channel having a second gain for processing a second processing signal derived from signals received via the sensor arrangement, the second gain being higher than the first gain.

Generally, the implantable medical device may be configured to process different processing signals. For obtaining such processing signals, a sensor arrangement is provided, the sensor arrangement comprising, e.g., one or multiple electrodes to receive electrical signals from which the processing signals are derived. The processing signals herein, for example, may be obtained each using a pair of electrodes, wherein for obtaining the different processing signals the same pair of electrodes or different pairs of electrodes may be used. In the first case, a single electrical signal, such as an intracardiac electrogram, may be obtained, from which different processing signals, namely the first processing signal and the second processing signal are derived for separate processing. In the latter case, separate electrical signals relating, for example, to a ventricular sensing signal and an atrial sensing signal (i.e., by applying a sensing optimized for atrial sensing) may be received in order to derive the first processing signal and the second processing signal from such different electrical signals, the different electrical signals, for example, being received using different pairs of electrodes of the sensor arrangement.

The different processing signals, in one embodiment, are processed in different processing paths of the processing circuitry. For this, the processing circuitry comprises a first processing channel for processing the first processing signal, the first processing signal relating, for example, to a near-field (in particular ventricular) sensing signal which, according to the placement of the implantable medical device, for example, in a ventricle of a patient's heart, may be strong such that the first processing channel may exhibit a rather low gain.

In addition, the processing circuitry comprises a second processing channel for processing the second processing signal, which may relate, for example, to a far-field atrial sensing signal which, in case of a placement of the implantable medical device in the ventricle, may have a small amplitude, due to the distance between the location of implantation and the source of origin of the signals. In order to allow for a reliable processing of the second processing signal, the second processing channel exhibits a gain higher than the gain of the first processing channel, such that features relating to an atrial activity may be suitably analyzed within the received signals.

Because, for a placement of the implantable medical device in, for example, the ventricle, atrial activity occurs in the far-field, atrial events within a regular ventricular sensing signal (for example, obtained be a regular ventricular QRS sensing channel) may be hard to discern, as a P wave stemming from atrial activity may exhibit a small amplitude in relation to QRS and T waves. For this reason, signal portions relating to far-field activity may be processed separately from signals relating to near-field activity within the second processing channel, such that within the second processing channel far-field events may be detected with increased reliability and enhanced timing precision.

The implantable medical device, in one aspect, is to be placed entirely or partially in the right or left ventricle.

In one aspect, the sensor arrangement is formed by an electrode arrangement, the electrode arrangement comprising a first electrode arranged in the vicinity of a tip of the body. The first electrode shall come to rest on cardiac tissue in an implanted state of the implantable medical device, such that the first electrode contacts cardiac tissue, e.g., at a location effective for injecting a stimulating signal into cardiac tissue for provoking a pacing action, in particular ventricular pacing.

In one aspect, the electrode arrangement comprises a second electrode formed by an electrode ring circumferentially extending about the body. Alternatively, the second electrode may, for example, be formed by a patch or another electrically conductive area formed on the body. The second electrode is placed at a distance from the tip of the body and hence at a distance from the first electrode arranged at the tip.

In one embodiment, the processing circuitry is configured to process, as said first processing signal, a first signal sensed between the first electrode and the second electrode. Such first signal may be denoted as near-field vector to be received between a pair of electrodes comprised of the first electrode and the second electrode. As the first electrode and the second electrode may, in one embodiment, be located at a rather close distance to each other, such pair of electrodes is predominantly suited to receive signals in close proximity to the implantable medical device, i.e., in the near-field region within the ventricle if the implantable medical device is implanted into the ventricle. The sense signal received in between the first electrode and the second electrode is provided to the first processing channel for processing in order to, for example, detect near-field (e.g., ventricular) events in the signal.

In one embodiment, the body comprises a remote location (e.g., the far end of a housing of a leadless pacemaker device) removed from the tip, the electrode arrangement comprising a third electrode arranged on the body at the remote location. The third electrode is operatively connected to the processing circuitry, such that the processing circuitry is enabled to receive and process signals received via the third electrode.

In one aspect, the processing circuitry is configured to process, as said second processing signal, a second signal sensed between the first electrode and the third electrode. Such second signal vector arising between the first electrode and the third electrode may be referred to as far-field vector, the first electrode and the third electrode exhibiting a distance with respect to each other larger than the first and the second electrode. The second signal may in particular be processed to detect events in the far-field, i.e., atrial contractions in case the implantable medical device is placed in the ventricle, such that by means of the second signal an intrinsic atrial activity prior to injecting a pacing stimulus may be captured.

The second signal sensed between the first electrode and the third electrode may be used to sense intrinsic atrial contractions in order to provide for an atrial-to-ventricular synchronization by timely injecting a stimulus at the ventricular location of implantation of the pacemaker device following atrial contractions. The second signal is provided to the second processing channel in order to process the signal and detect atrial events from the signal, in order to provide for a pacing action based on detected atrial events, hence allowing for a ventricular pacing with atrioventricular (AV) synchrony.

In one embodiment, the second processing channel comprises a processing stage for differentiating one wave portion from another wave portion in the second processing signal. The processing stage in particular may be configured to apply, to the second processing signal, at least one of a bandpass filtering, a blanking window for excluding a portion of the second signal from further processing, a moving average filtering, and a rectification. By means of the processing stage, in particular such wave portions shall be isolated and/or emphasized within the signal to be processed which may be indicative of, e.g., an atrial event. If the implantable medical device is placed in the ventricle of a patient's heart, signal portions relating to far-field atrial activity may have a much smaller amplitude than signal portions relating to a near-field ventricular activity. Hence, the processing serves to differentiate between the different signal portions in order to identify such signal portion which may contain signals relating to far-field atrial activity.

For isolating, e.g., the P wave in an intracardiac electrogram, a bandpass filtering may be applied, hence differentiating wave portions relating to the P wave from wave portions in particular relating to QRS and T waves stemming from ventricular activity. Alternatively or in addition, a blanking method may be applied in order to blank out certain portions of the second processing signal, namely such portions which contain signals stemming from events other than a far-field atrial activity. A blanking window, for this, serves to silence signal portions which are not of interest for far-field activity, but which may rather interfere with the detection of far-field activity. By means of a blanking window such portions of the signal which do not relate to far-field atrial activity hence are excluded from processing, such that the processing is limited to those signal portions (likely) relating to far-field activity. Alternatively or in addition, other methods such as a moving average filtering, finite differences or a rectification of the signal may be applied. A moving averaging filter herein can be used to smooth the processing signal. Rectification can serve to easily compare the processed signal to a (single) threshold in order to identify when the signal magnitude exceeds a predefined threshold.

In another aspect, a method for operating an implantable medical device for providing for an intracardiac function comprises: receiving, using a sensor arrangement arranged on a body of the implantable medical device, cardiac sense signals; and processing, using a processing circuitry operatively connected to the sensor arrangement, cardiac sense signals received using the sensor arrangement to detect, in a cardiac cycle, a first atrial event candidate based on a comparison of the cardiac sense signal with a sense threshold, to determine a modified sense threshold, to monitor, by comparing the cardiac sense signal to said modified sense threshold following the detection of the first atrial event candidate, whether in the same cardiac cycle a second atrial event candidate is detected.

The advantages and advantageous embodiment described above for the device equally apply also to the method, such that is shall be referred to the above.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The various features and advantages of the present invention may be more readily understood with reference to the following detailed description and the embodiments shown in the drawings. Herein,

FIG. 1 shows a schematic view of the human heart, with an implantable medical device in the shape of a leadless pacemaker device implanted therein;

FIG. 2 shows a schematic view of an implantable medical device;

FIG. 3 shows a schematic view of an implantable medical device, indicating signal vectors between different electrodes of the implantable medical device;

FIG. 4 shows a schematic view of a processing circuitry of an embodiment of an implantable medical device;

FIG. 5A shows a first processing signal in the shape of an intracardiac electrogram (IEGM) processed by a first processing channel of the processing circuitry;

FIG. 5B shows a second processing signal processed by a second processing channel of the processing circuitry;

FIG. 6A shows a first processing signal in the shape of an intra-cardiac electrogram (IEGM) processed by the first processing channel of the processing circuitry;

FIG. 6B shows a second processing signal processed by the second processing channel; and

FIG. 7 shows a schematic view of the human heart, with an implantable medical device in the shape of cardiac stimulation device having a lead implanted in the right ventricle.

### DETAILED DESCRIPTION

Subsequently, embodiments of the present invention shall be described in detail with reference to the drawings. In the drawings, like reference numerals designate like structural elements.

It is to be noted that the embodiments are not limiting for the present invention, but merely represent illustrative examples.

In the instant invention it is proposed to provide an implantable medical device providing for an intracardiac function, in particular a ventricular pacing, specifically a so-called VDD pacing.

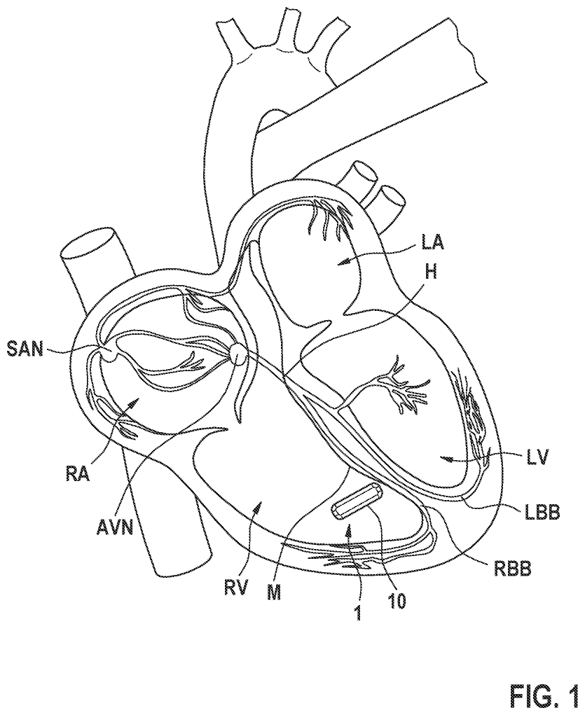

FIG. 1 shows, in a schematic drawing, the human heart comprising the right atrium RA, the right ventricle RV, the left atrium LA and the left ventricle LV, the so-called sinoatrial node SAN being located in the wall of the right atrium RA, the sinoatrial node SAN being formed by a group of cells having the ability to spontaneously produce an electrical impulse that travels through the heart's electrical conduction system, thus causing the heart to contract in order to pump blood through the heart. The atrioventricular node AVN serves to coordinate electrical conduction in between the atria and the ventricles and is located at the lower back section of the intra-atrial septum near the opening of the coronary sinus. From the atrioventricular node AVN the so-called HIS bundle H is extending, the HIS bundle H being comprised of heart muscle cells specialized for electrical conduction and forming part of the electrical conduction system for transmitting electrical impulses from the atrioventricular node AVN via the so-called right bundle branch RBB around the right ventricle RV and via the left bundle branch LBB around the left ventricle LV.

In case of a block at the atrioventricular node AVN, the intrinsic electrical conduction system of the heart H may be disrupted, causing a potentially insufficient intrinsic stimulation of ventricular activity, i.e., insufficient or irregular contractions of the right and/or left ventricle RV, LV. In such a case, a pacing of ventricular activity by means of a pacemaker device may be indicated, such pacemaker device stimulating ventricular activity by injecting stimulation energy into intracardiac tissue, specifically myocardium M.

In one embodiment, an implantable medical device 1 in the shape of a leadless cardiac pacemaker device, as schematically indicated in FIG. 1, is provided for a ventricular pacing action, the leadless pacemaker device having a body 10 formed by the housing of the leadless pacemaker device.

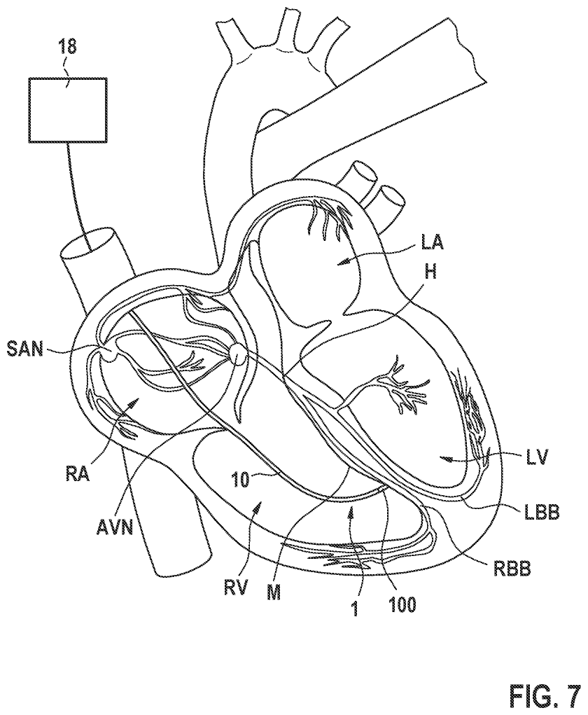

In another embodiment, as shown in FIG. 7, the implantable medical device 1 may be a stimulation device having a generator 18 and at least one lead forming a body 10 of the implantable medical device 1 and extending transvenously from the generator 18 into the patient's heart.

Whereas common implantable medical devices are designed to sense a ventricular activity by receiving electrical signals from the ventricle RV, LV they are placed in, it may be desirable to provide for a pacing action which achieves atrioventricular (AV) synchrony by providing a pacing in the ventricle in synchrony with an intrinsic atrial activity. For such pacing mode, also denoted as VDD pacing mode, it is required to sense atrial activity and identify atrial events relating to atrial contractions in order to base a ventricular pacing on such atrial events.

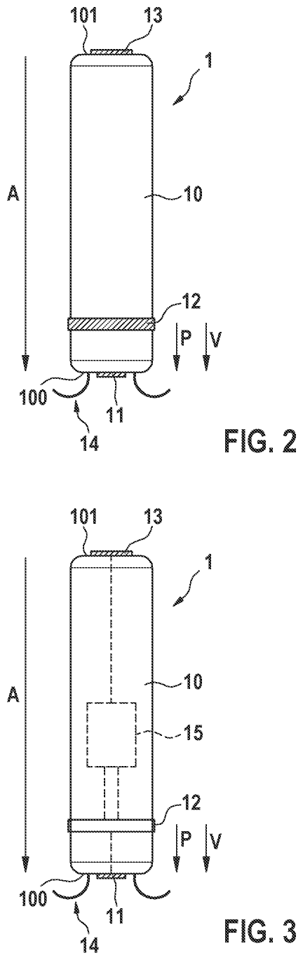

Referring now to FIGS. 2 and 3, in one embodiment an implantable medical device 1 in the shape of a leadless pacemaker device configured to provide for an intracardiac pacing, in particular in a VDD pacing mode, comprises a housing 10 enclosing electrical and electronic components for operating the implantable medical device 1. In particular, enclosed within the housing 10 is a processing circuitry 15, comprising, for example, also a communication interface for communicating with an external device, such as a programmer wand. In addition, electrical and electronic components such as an energy storage in the shape of a battery are confined in the housing 10. The housing 10 provides for an encapsulation of components received therein, the housing 10 having the shape of, e.g., a cylindrical shaft having a length of, for example, a few centimeters.

The implantable medical device 1 is to be implanted immediately on intracardiac tissue M. For this, the implantable medical device 1 comprises, in the region of a tip 100, a fixation device 14, for example, in the shape of nitinol wires to engage with intracardiac tissue M for fixedly holding the implantable medical device 1 on the tissue in an implanted state.

The implantable medical device 1 in the embodiment of FIGS. 2 and 3 does not comprise leads, but receives signals relating to a cardiac activity by means of an electrode arrangement arranged on the housing 10 and also emits stimulation signals by means of such electrode arrangement. In the embodiment of FIGS. 2 and 3, the implantable medical device 1 comprises different electrodes 11, 12, 13 making up the electrode arrangement and serving to emit pacing signals towards intra-cardiac tissue M for providing a pacing and to sense electrical signals indicative of a cardiac activity, in particular indicative of atrial and ventricular contractions.

A first electrode 11 herein is denoted as pacing electrode. The first electrode 11 is placed at a tip 100 of the housing 10 and is configured to engage with cardiac tissue M.

A second electrode 12 herein is denoted as pacing ring. The second electrode 12 serves as a counter-electrode for the first electrode 11, a signal vector P arising between the first electrode 11 and the second electrode 12 providing for a pacing vector P for emitting pacing signals towards the intra-cardiac tissue M.

In addition, the second electrode 12 serves as a sensing electrode for sensing signals, in particular relating to ventricular contractions, a signal vector V arising between the second electrode 12 and the first electrode 11, the signal vector V being denoted as near-field vector.

The second electrode 12 is placed at a distance from the first electrode 11 and, for example, has the shape of a ring extending circumferentially about the housing 10. The second electrode 12 is, for example, placed at a distance of about 1 cm from the tip 100 of the housing 10 at which the first electrode 11 is placed.

The implantable medical device 1, in the embodiment of FIGS. 2 and 3, in addition comprises a third electrode 13 placed at a far end 101 of the housing 10, the third electrode 13 serving as a sensing electrode for sensing signals indicative of cardiac activity in the far-field. In particular, a signal vector A arises between the third electrode 13 and the first electrode 11, the signal vector A picking up signals being indicative, for example, of atrial contractions and being denoted as far-field vector.

The electrodes 11, 12, 13 are in operative connection with the processing circuitry 15, the processing circuitry 15 being configured to cause the first electrode 11 and the second electrode 12 to emit a pacing signal for providing a stimulation at the ventricle. The processing circuitry 15 furthermore is configured to process signals received via the electrodes 11, 12, 13 to provide for a sensing of cardiac activity, in particular atrial and ventricular contractions.

If the implantable medical device 1 has the shape of a stimulation device comprising a generator 18 and a lead extending from the generator 18, as shown in the embodiment of FIG. 7, a similar electrode arrangement comprising, for example, three electrodes 11, 12, 13 may be arranged on a lead implanted in and extending into the right ventricle RV, as shown in FIG. 7, such that the above also applies to an embodiment of the implantable medical device 1 having a lead extending into the patient's heart. In this case, the processing circuitry may be part of the generator 18 and may be in operative connection with an electrode arrangement arranged on the lead.

In order to provide for a pacing in the ventricle in which the implantable medical device 1 is placed, in particular to enable a pacing in the VDD mode, a sensing of atrial activity is required to provide for detected atrial sense markers in order to time a pacing in the ventricle to obtain atrioventricular (AV) synchrony. For this, a far-field signal from in particular the right atrium RA (see FIGS. 1 and 9) shall be sensed in order to allow for a synchronous pacing in the right ventricle RV by means of the implantable medical device 1 being implanted on intracardiac tissue M in the right ventricle RV.

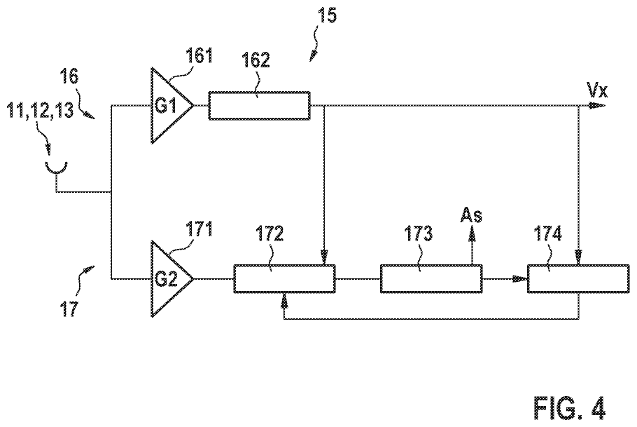

Referring now to FIG. 4, the processing circuitry 15 comprises, in one embodiment, two processing channels 16, 17 for processing different processing signals relating to ventricular activity and atrial activity. Herein, typically, an intracardiac electrogram (IEGM) contains a signal portions relating to ventricular activity (in particular a QRS wave) and atrial activity (in particular a P wave), signal portions relating to atrial activity however resulting from a far-field signal source and hence being far less pronounced and having a far smaller amplitude then signal portions relating to a ventricular activity in the near-field, i.e., arising in close proximity to the implanted implantable medical device 1. For this reason, the two processing channels 16, 17 are associated with different gains G1, G2, a first processing channel 16 serving to process a first processing signal to identify ventricular events at a rather low gain G1 and a second processing channel 17 being configured to process a second processing signal to identify atrial events at a significantly higher gain G2.

In particular, the first processing channel 16 is connected to the electrode arrangement comprised of the electrodes 11, 12, 13, the first processing channel 16 being configured in particular to sense and process a signal received via the electrodes 11, 12 (near-field vector V in FIGS. 2 and 3). The first processing channel 16 comprises a first amplification stage 161 having a gain G1 and, following the amplification stage 161, a detection stage 162 which is configured to identify ventricular sense markers Vx from the first processing signal processed within the first processing channel 16.

The second processing channel 17 is likewise connected to the electrode arrangement comprised of electrodes 11, 12, 13, wherein the second processing channel 17 may in particular be configured to process a signal sensed via the far-field vector A, that is in between the electrodes 11, 13 placed at the tip 100 and the far end 101 of the housing 10 as illustrated in FIGS. 2 and 3. The second processing channel 17 comprises a second amplification stage 171 having a second gain G2, the second amplification stage 171 being followed by a processing stage 172 and a second detection stage 173.

The processing stage 172 serves to pre-process the second processing signal after amplification. The detection stage 173 in turn serves to evaluate and analyze the processed signal in order to identify atrial events within the second processing signal, the second processing channel 17 then outputting atrial sense markers As indicative of atrial events detected in the processed signal.

In addition, the processing circuitry 15 comprises a timing stage 174 which uses timing information received from the first processing channel 16 and the second processing channel 17 to provide for a pacing timing, in particular a VDD timing for achieving an atrial-ventricular synchronous pacing.

In order to identify and analyze atrial events, the gain G2 of the second processing channel 17 is (significantly) higher than the gain G1 of the first processing channel 16. This generally allows to analyze signal portions relating to atrial events, but makes it necessary to discern such signal portions relating to atrial events from other signal portions, in particular signal portions relating to ventricular events in the near-field and hence being far stronger than signal portions originating from atrial events in the far-field.

Within the processing stage 172, for example, a bandpass filtering, a windowing (e.g., partial blanking), a smoothing by means of a moving average filtering and a rectification may take place. A first or second order difference may be applied to remove a non-zero baseline while enhancing P wave defections.

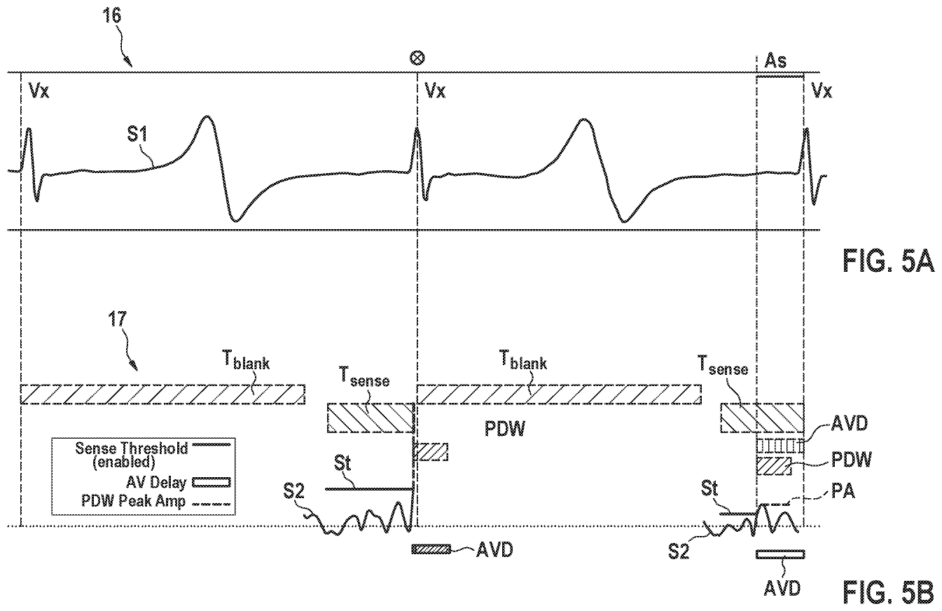

FIGS. 5A and 5B show examples of signals S1, S2 as processed in the different processing channels 16, 17, FIG. 5A at the top showing a signal S1 as processed by the first processing channel 16 and FIG. 5B at the bottom showing a signal S2 as processed by the second processing channel 17. As a result of the processing, ventricular events Vx and atrial events As are identified and corresponding markers are output.

As apparent from FIG. 5B, the sensing of atrial events As uses a windowing scheme, employing in particular a blanking window T_{blank} for blanking out signal portions of the signal S2 potentially relating to ventricular activity.

In particular, by means of the detection of ventricular events Vx in the first processing channel 16 a timing in between atrial events As and ventricular events Vx may be determined. According to such timing a start point and an end point of the blanking window T_{blank} may be set, hence excluding signal portions from the processing which do not relate to atrial activity. Strong ventricular signals in this way may be suppressed such that signal portions relating to a ventricular activity may not interfere with a detection of atrial events.

During the blanking window T_{blank}, the second processing channel 17 may be turned off. In particular, the amplification stage 171 of the second processing channel 17 may be switched of in order to save power.

Generally, a detection for atrial events takes place outside of the blanking window T_{blank}. Herein, a detection window Tₛₑₙₛₑ for detecting atrial events may start at the end of a prior blanking window T_{blank}. Alternatively, a detection window Tₛₑₙₛₑ may—as shown in the embodiment of FIG. 5B—have a delay with respect to the end of a prior blanking window T_{blank}, such that a signal processing within the second processing channel 17 starts at the end of a prior blanking window T_{blank}, a detection for atrial events however starting only after a certain delay.

Generally, an atrial event As is assumed to be present if, in the detection window Tₛₑₙₛₑ, the signal S2 crosses a sense threshold ST, as it is shown in FIG. 5B. The comparison may take place based on a rectification of the sense signal S2. Alternatively, a positive and negative sense threshold ST may be used, which may have the same value or may differ in their values. A threshold crossing herein may be assumed if one signal value is larger than the sense threshold ST. Alternatively, a crossing of the sense threshold ST is assumed if a predefined number of signal values are larger than the sense threshold ST, for example, two or more consecutive sample values.

Generally, if an atrial event As is detected, as it is the case for the second cardiac cycle in FIG. 5B, the atrial event As is used for a further processing, in particular to update the sense threshold ST and to achieve an atrial-ventricular synchronous pacing.

In particular, the atrial event As is taken as that point in time at which a crossing of the sense threshold ST is identified. At the time of the atrial event As a peak detection window PDW starts, and based on data recorded during that peak detection window PDW a peak amplitude PA is determined as the maximum signal value within the peak detection window PDW. This is indicated in FIG. 5B for the second cycle at the right.

Also, in case of a detection of an atrial event As, an atrial-ventricular delay AVD may be determined and used for subsequent processing. If no ventricular event Vx is detected after lapse of the atrial-ventricular delay AVD, a pacing signal may be injected to cause a ventricular stimulation.

The peak amplitude PA, in one embodiment, may be used to update the sense threshold ST for the next cardiac cycle. In particular, the processing circuitry 15 may be configured to update the sense threshold ST using an average threshold reference and a percentage ratio according to the formula

ST=PC·ATR(t),

where ST is the current sense threshold, PC is the percentage ratio, and ATR(t) is the average threshold reference for the current cycle t. The percentage ratio may lie, for example, in the range between 0% and 100%.

The average threshold reference may be determined based on a mean value for a number of previous cardiac cycles in which atrial events have been identified and correspondingly peak amplitude values have been obtained. The average threshold reference in this case, for example, may be determined as the average of the peak amplitude values in the previous cardiac cycles.

In another embodiment, the average threshold reference may be computed based on the peak amplitude PA according to the following equation:

ATR(t)=W·PA(t−1)+(1−W)·ATR(t−1),

where W indicates an update weight which determines how much the average threshold reference should change based on the previous peak amplitude, PA(t−1) is the peak amplitude as determined for the previous cycle t−1, and ATR(t−1) is the previous average threshold reference. For the actual cycle t the average threshold reference hence is determined based on the previously determined valid peak amplitude and on the previous average threshold reference for cycle t−1. For each cycle, hence, the average threshold reference is updated and computed anew, such that the average threshold reference is dynamically adjusted on a cycle-by-cycle basis.

For the actual cycle t the average threshold reference hence is determined based on the peak amplitude PA determined for that cycle t and based on the previously valid average threshold reference at cycle t−1. For each cycle for which an atrial event As is detected, hence, the average threshold reference is updated and computed anew, such that the average threshold reference is dynamically adjusted on a cycle-by-cycle basis.

If no (valid) atrial event As is detected, no peak amplitude PA is determined and the sense threshold ST is not updated. In this way it is avoided that a false detection of an atrial event As may cause a false increase of the sense threshold ST and a subsequent capture loss of atrial activity. This is the case for the first cardiac cycle as shown in FIGS. 5A and 5B, in which no crossing of the sense threshold ST is detected and correspondingly no atrial event As is identified.

The sense threshold ST generally is set based on the peak amplitude value PA of detected atrial events As in previous cardiac cycles. Correspondingly, dependent on the peak amplitude values PA in the previous cardiac cycles the sense threshold ST may be dynamically raised or lowered.

Generally, as shown in FIGS. 5A and 5B, an atrial event As is identified based on a comparison of a cardiac sense signal, for example, an intra-cardiac electrogram signal S2, to the sense threshold ST valid at that time for the cardiac cycle. As a cardiac sense signal relating to atrial activity is received in the far-field if the implantable medical device 1 is implanted with its sensor arrangement in the ventricle, for example, the right ventricle, of the patient's heart, the signal may be weak and noisy, such that it may be hard to discern a true atrial event from other signal deflections, which potentially also may cause a threshold crossing. In particular, signal deflections prior to an actual atrial event may cause a threshold crossing and may be erroneously identified as an atrial event, potentially leading to a incorrect timing for a pacing action and hence to a loss of atrial-ventricular synchrony.

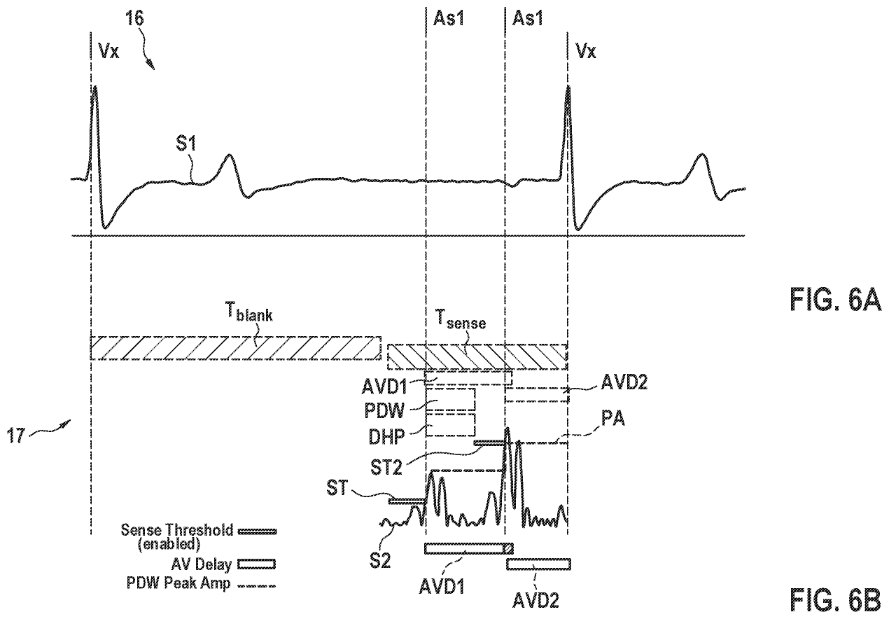

Referring now to FIGS. 6A and 6B, in order to improve reliability of identification of an atrial event As within an atrial detection window Tₛₑₙₛₑ a scheme is proposed in which an initial detection of a first atrial event candidate As1 is verified by a subsequent monitoring for further atrial event candidates As2.

For this, the cardiac sense signal S2 initially is compared to a sense threshold ST, as is set, for example, according to an average threshold reference based on peak amplitude values PA of prior cardiac cycles. If a threshold crossing is identified, at the time of the threshold crossing a first atrial event candidate As1 is assumed to be detected, as illustrated in FIGS. 6A and 6B. The atrial event As1 herein may be assumed to be detected if one sample value is larger than the sense threshold ST, or if a predefined number of multiple sample values are recorded to be larger than the sense threshold ST.

At the time of the first atrial event candidate As1, a peak detection window PDW is started, and within the peak detection window PDW the (rectified) sense signal S2 is tracked in order to determine the maximum value of the sense signal S2 within the peak detection window PDW. The maximum value sets the peak amplitude PA.

In addition, a detection hold-off period DHP is started at the time of the first atrial event candidate As1, no monitoring of further atrial event candidates takes place within the detection hold-off period.

Based on the peak amplitude PA as determined within the peak detection window PDW a modified sense threshold ST2 is determined, which may be computed, for example, by multiplying the peak amplitude PA as determined in the peak detection window PDW by a pre-defined factor (>1), such that the modified sense threshold ST2 is larger than the peak amplitude PA (and also is larger than the initial sense threshold ST).

Using the modified sense threshold ST2 it is monitored whether the sense signal S2 crosses the modified sense threshold ST2 after lapse of the detection hold-off period. If such threshold crossing is found, a second atrial event candidate As2 is identified, and in the shown embodiment the peak amplitude PA is set to the value of the signal S2 at the time of the second atrial event candidate As2.

In one embodiment, it only is monitored for two atrial event candidates As1, As2. In another embodiment it may be monitored for further atrial event candidates following the second atrial event candidate As2.

The peak amplitude PA may be set to the value of the signal S2 at the time of the second atrial event candidate As2. Alternatively, anew peak detection window PDW may be started at the time of the second atrial event candidate As2, and the signal S2 may be tracked in order to determine the maximum value of the signal S2 within the peak detection window PDW to set the peak amplitude PA. In yet another embodiment, the peak detection window PDW as started at the detection of the first atrial event candidate As1 may have a length to substantially cover all signal peaks within the detection window Tₛₑₙₛₑ, such that a single, prolonged peak detection window PDW runs during the detection window Tₛₑₙₛₑ to determine the maximum signal value within the detection window Tₛₑₙₛₑ.

The detection hold-off period DHP and the peak detection window PDW may be programmable in their length. The detection hold-off period DHP and the peak detection window PDW may be equal in length or may have different lengths.

At the time of the first atrial event candidate As1 an atrial-ventricular delay AVD1 is started. The monitoring for a subsequent second atrial event candidate As2 in the same cardiac cycle takes place only while the atrial-ventricular delay AVD1 has not expired, the atrial-ventricular delay AVD1 hence indicating a time limit for the monitoring for a further atrial event candidate As2.

If a second atrial event candidate As2 is detected, another, new atrial-ventricular delay AVD2 is started.

If, as in the example of FIGS. 6A, 6B, a second atrial event candidate As2 is detected subsequent to a first atrial event candidate As1, the second atrial event As2 is assumed to be a correct atrial event corresponding to a P wave in the sense signal S2, and an atrial sense marker As is identified and output for the second atrial event As2. The first atrial event candidate As1 hence is overridden and discarded. The peak amplitude PA is set according to the second atrial event As2, which now is assumed to be the true atrial event.

Also, using the atrial-ventricular delay AVD2 associated with the second atrial event candidate As2, now a further processing takes place, in particular a potential triggering of a pacing action in case no ventricular activity is found after lapse of the atrial-ventricular delay AVD2.

From FIGS. 6A, 6B it is apparent that a further, second atrial event As2 is detected only if its signal amplitude is larger than a prior, first atrial event As1. This is due to the setting of the modified sense threshold ST2, which is larger than the peak amplitude PA as derived from the maximum signal value of the first atrial event As1.

Using atrial sense markers As output by the processing circuitry 15, a ventricular synchronous pacing may be achieved. For this, it can be detected whether, following a detected atrial sense marker As, a ventricular event Vx (intrinsic ventricular sense or ventricular pace) occurs within a predefined time delay window (corresponding to the atrial-ventricular delay AVD, AVD1, AVD2) after the atrial sense marker As. If no intrinsic ventricular sense marker is detected, a stimulation pulse may be emitted, causing a synchronous pacing in the ventricle.

Conversely, also an asynchronous pacing can be performed.

Utilizing a far-field electrical signal received by means of an implantable medical device can offer a superior detection of far-field events, in particular atrial events in case the implantable medical device is implanted into the ventricle. Tracking of far-field events by using and evaluating electrical signals may allow for increased consistency and reliability in particular with respect to external factors such as posture and patient activity.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 Implantable medical device (leadless pacemaker device)
10 Body (housing)
100 Tip
101 Far end
11 First electrode (pacing electrode)
12 Second electrode (pacing ring)
13 Third electrode
14 Fixation device
15 Processing circuitry
16 First processing channel
161 Amplification stage
162 Detection stage
17 Second processing channel
171 Amplification stage
172 Processing stage
173 Detection stage
174 Timing stage
18 Generator
A Atrial signal vector
As, As1, As2 Atrial event (candidate)
ATR Average threshold reference
AVD Atrial-ventricular delay
AVD1, AVD2 Atrial-ventricular delay
AVN Atrioventricular node
DHP Detection hold-off period
G1, G2 Gain
H HIS bundle
LA Left atrium
LAT Lower absolute threshold
LBB Left bundle branch
LV Left ventricle
M Intra-cardiac tissue (myocardium)
P Pacing signal vector
PA Peak amplitude
PDW Peak detection window
RA Right atrium
RBB Right bundle branch
RV Right ventricle
S1, S2 Signal
SAN Sinoatrial node
ST, ST2 Sense threshold
T_{blank} Blanking window
Tₛₑₙₛₑ Detection window
V Ventricular signal vector
Vx Ventricular event

1. An implantable medical device configured to provide for an intracardiac function, the implantable medical device comprising:
a body;
a sensor arrangement arranged on the body and configured to receive a cardiac sense signal; and
a processing circuitry operatively connected to the sensor arrangement, wherein the processing circuitry is configured to process the cardiac sense signal received using the sensor arrangement
to detect, in a cardiac cycle, a first atrial event candidate based on a comparison of the cardiac sense signal with a sense threshold,
to determine a modified sense threshold,
to monitor, by comparing the cardiac sense signal to said modified sense threshold following the detection of the first atrial event candidate, whether in the same cardiac cycle a second atrial event candidate is detected.

2. The implantable medical device according to claim 1, wherein the body is formed by a lead which is connectable to a generator of the implantable medical device, or wherein the body is formed by a housing of a leadless pacemaker device.

3. The implantable medical device according to claim 1, wherein the processing circuitry is configured to identify, if a second atrial event candidate is detected, the second atrial event candidate as valid atrial event for that cardiac cycle.

4. The implantable medical device according to claim 1, wherein the processing circuitry is configured to identify the first atrial event candidate as valid atrial event for the cardiac cycle if no second atrial event candidate is detected.

5. The implantable medical device according to claim 1, wherein the processing circuitry is configured to determine a new modified sense threshold if a second atrial event candidate is detected, and to monitor, by comparing the cardiac sense signal to said new modified sense threshold following the detection of the second atrial event candidate, whether in the same cardiac cycle a third atrial event candidate is detected.

6. The implantable medical device according to claim 1, wherein the processing circuitry is configured to detect said first atrial event candidate based on a crossing of the sense threshold by said cardiac sense signal.

7. The implantable medical device according to claim 1, wherein the processing circuitry is configured to determine said modified sense threshold based on a peak amplitude determined for the first atrial event candidate.

8. The implantable medical device according to claim 7, wherein the processing circuitry is configured to determine the peak amplitude in a peak detection window following said first atrial event candidate.

9. The implantable medical device according to claim 7, wherein the processing circuitry is configured to set, if a second atrial event candidate is detected, the peak amplitude anew according to a signal value associated with the second atrial event candidate.

10. The implantable medical device according to claim 1, wherein the processing circuitry is configured to detect said second atrial event candidate based on a crossing of the modified sense threshold by said cardiac sense signal.

11. The implantable medical device according to claim 1, wherein the processing circuitry is configured to start, at the time of detection of the first atrial event candidate, a detection hold-off period and to delay monitoring for the detection of said second atrial event candidate until after lapse of said detection hold-off period.

12. The implantable medical device according to claim 1, wherein the processing circuitry is configured to start, at the time of detection of the first atrial event candidate, an atrial-ventricular delay period and to stop monitoring for the detection of said second atrial event candidate after lapse of said atrial-ventricular delay period.

13. The implantable medical device according to claim 12, wherein the processing circuitry is configured to start, at the time of detection of said second atrial event candidate, the atrial-ventricular delay period anew.

14. The implantable medical device according to claim 1, wherein the processing circuitry comprises a first processing channel having a first gain for processing a first processing signal derived from cardiac sense signals received via the sensor arrangement and a second processing channel having a second gain for processing a second processing signal derived from cardiac sense signals received via the sensor arrangement, the second gain being higher than the first gain.

15. Method for operating an implantable medical device for providing for an intra-cardiac function, comprising:
receiving, using a sensor arrangement arranged on a body of the implantable medical device, cardiac sense signals; and
processing, using a processing circuitry operatively connected to the sensor arrangement, cardiac sense signals received using the sensor arrangement
to detect, in a cardiac cycle, a first atrial event candidate based on a comparison of the cardiac sense signal with a sense threshold,
to determine a modified sense threshold,
to monitor, by comparing the cardiac sense signal to said modified sense threshold following the detection of the first atrial event candidate, whether in the same cardiac cycle a second atrial event candidate is detected.
